(19) United States
(12) Patent Application Publication
(10) Pub. No.: US 2016/0046572 A1
(43) Pub. Date: Feb. 18, 2016

(54) Process for Preparing Sulfimines and Their in-situ Conversion into N-(1-Amino-Benzoyl)-Sulfimines

(71) Applicant: BASF SE, Ludwigshafen (DE)

(72) Inventors: Timo FRASSETTO, Mannheim (DE); Harald JOCKERS, Ludwigshafen (DE); Christopher KORADIN, Gommersheim (DE); Thomas ZIERKE, Hassloch (DE); Karsten KOERBER, Eppelheim (DE)

(73) Assignee: BASF SE, Ludwigshafen (DE)

(21) Appl. No.: 14/778,771

(22) PCT Filed: Mar. 27, 2014
(86) PCT No.: PCT/EP2014/056164

§ 371 Date: Sep. 21, 2015

(30) Foreign Application Priority Data
Mar. 28, 2013 (EP) 13161739.1

(51) Int. Cl. C07C 381/10; C07D 401/04

(52) U.S. Cl. CPC C07C 381/10; C07D 401/04

(57) ABSTRACT

The present invention relates to a process for preparing a compound of the formulae (Ia) or (Ib), or a mixture thereof,

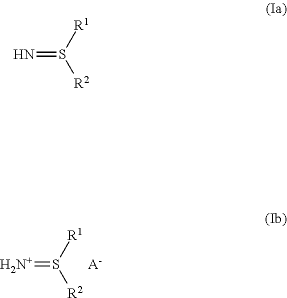

wherein
R¹ and R² independently of one another are hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl or together represent an aliphatic chain, or the like; A⁻ is HSO₄⁻ or 1/2 SO₄²⁻;
the process comprising the reaction of a sulfide of formula SR¹R² with hydroxylamineO-sulfonic acid of formula; wherein the reaction is carried out in an aqueous medium in the presence of a base.

The present invention also relates to a process for preparing a compound of the formula (IV),

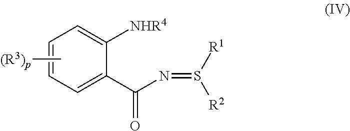

wherein
R³ is halogen, cyano, C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₁-C₈-alkoxy, phenyl, or the like; R⁴ is hydrogen, C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, phenyl, or the like; p is 0, 1, 2, 3 or 4;
the process comprising:
(i) providing the compound of the formulae (Ia) or (Ib), or a mixture thereof,
(ii) reacting a compound of the formulae (Ia) or (Ib), or a mixture thereof, obtained in step (i) with a compound of the formula (V) in the presence of a base,

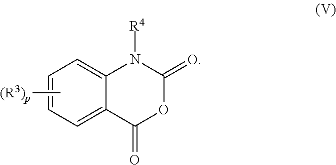

The present invention relates to a process for preparing sulfimines NH═SR¹R² or their sulfates or hydrogensulfates, wherein R¹ and R² are as defined hereinafter and in the claims. The process includes the reaction of the corresponding sulfides with hydroxylamine-O-sulfonic acid in an aqueous medium in the presence of a base. The invention also relates to the in-situ conversion of the thus obtained sulfimines or its (hydrogen)sulfates into the corresponding N-(2-amino-benzoyl)-sulfimines.

N-(2-Amino-benzoyl)-sulfimines are of great interest especially because they are crucial precursors for the highly effective process disclosed in WO 2013/024008 for the preparation of N-(het)arylpyrazole carboxanilides carrying a sulfiminocarbonyl group in the ortho position on the anilide moiety. These compounds belong to the class of anthranilamide insecticides, for which cyantraniliprole and chlorantraniliprole are prominent examples, and have been described in WO 2007/006670 to show high activity against invertebrate pests.

N-Unsubstituted sulfimines, such as of formula NH═SR¹R², or their salts are typically prepared by S-amination of thioethers using as amination reagent hydroxylamine-O-sulfonic acid (see for example R. Appel et al., Liebigs Annalen 1958, 618, 53; Angew. Chem. 1959, 71, 701; and Ber. Dtsch. Chem. Ges. 1962, 95, 849) or O-(mesitylene-sulfonyl)-hydroxylamine (see for example Y. Tamura et al., J. Org. Chem. 1973, 38, 4324) or similar reagents. All these procedures are carried out by reacting a thioether with the amination reagent in a non-aqueous medium and, in the case of hydroxylamine-O-sulfonic acid, also in the presence of sodium methoxide as base. Thus, for the latter reaction aqueous solvents have been avoided already because R. Appel et al., Ber. Dtsch. Chem. Ges. 1962, 95, 855, teaches that hydroxyl ions lead to the rapid hydrolytic degradation of N-unsubstituted sulfimines.

The aforementioned processes of the prior art using hydroxylamine-O-sulfonic acid suffer from the very high energy content of this reagent which presents a safety hazard particularly in conversions on an industrial scale.

According to WO 2013/024008 N-(2-amino-benzoyl)-sulfimines are prepared by reacting isatoic anhydrides with N-unsubstituted sulfimines or their salts in the presence of a base in a non-aqueous medium. The sulfimines or their salts are employed in these reactions as isolated products that often even require further purification, e.g. by crystallization. Thus, the known route for preparing N-(2-amino-benzoyl)-sulfimines is tedious and time consuming as it requires two separate reactions steps and the in-between isolation and possibly purification of the N-unsubstituted sulfimines or their salts.

Therefore, it is the object of the present invention to provide an economically attractive and technically feasible process that allows the preparation of N-(2-amino-benzoyl)sulfimines starting from sulfides, such as thioethers, and hydroxylamine-O-sulfonic acid. The process should be easy to perform and be suitable for industrial scale production. In addition, it should minimize the hazard presented by hydroxylamine-O-sulfonic acid.

The object is achieved by the processes described in detail below.

In a first aspect the present invention relates to a process for preparing a compound of the formulae (Ia) or (Ib), or a mixture thereof,

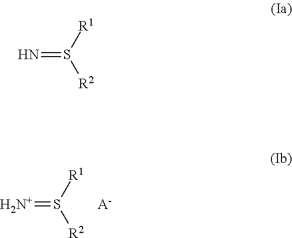

wherein
R¹ and R² are selected, independently of one another, from the group consisting of hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a},
or R¹ and R² together represent a C₂-C₉-alkylene, C₂-C₉-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₉-alkylene chain or 1 to 4 of any of the CH₂ or CH groups in the C₂-C₉-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NR^{y}, and wherein the carbon atoms in the C₂-C₉-alkylene, C₂-C₉-alkenylene or C₆-C₉-alkynylene chain may be substituted with 1 to 5 identical or different substituents R^{x}, and wherein the sulfur and nitrogen atoms in the C₂-C₉-alkylene, C₂-C₉-alkenylene or C₆-C₉-alkynylene chain, independently of one another, may be oxidized,
A⁻ is HSO₄⁻ or 1/2 SO₄²⁻,
R^{a} is selected from the group consisting of cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R^{f})₂R^{g}, —OR^{b}, —SR^{b}, —S(O)ₘR^{b}, —S(O)ₙN(R^{c})R^{d}, —N(R^{c})R^{d}, —C(═O)R^{b}, C(═O)OR^{b}, C(═O)N(R^{c})R^{d}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e},
or two geminally bound radicals R^{a} together form a group selected from ═CR^{h}R^{k}, ═NR^{c}, ═NOR^{b} and ═NNR^{c},
or two radicals R^{a}, together with the carbon atoms to which they are bound, form a 3-, 4-, 5-, 6-, 7- or 8-membered saturated or partially unsaturated carbocyclic ring or a 3-, 4-, 5-, 6-, 7- or 8-membered saturated or partially unsaturated heterocyclic ring containing 1, 2 or 3 heteroatoms or heteroatom groups selected from N, O, S, NO, SO and SO₂, as ring members,
wherein, in the case of more than one R^{a}, R^{a} can be identical or different,
R^{b} is selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, where the five last mentioned radicals may be unsubstituted, partially or fully halogenated and/or wherein one or two CH₂ groups may be replaced by a CO group; and/or may carry 1-2 radicals selected from C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxycarbonyl, —Si(R^{f})₂R^{g}, phenyl, benzyl, pyridyl and phenoxy, it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
wherein, in the case of more than one R^{b}, R^{b} can be identical or different,
R^{c}, R^{d} are, independently from one another, selected from the group consisting of hydrogen, cyano, C₂-C₆-alkyl, C₂-C₆-alkenyl, C₂-C₆-alkynyl, C₃-C₈-cycloalkyl, C₃-C₈-cycloalkyl-C₁-C₄-alkyl, where the five last mentioned radicals may be unsubstituted, partially or fully halogenated and/or wherein one or two CH₂ groups may be replaced by a CO group; and/or may carry 1 or 2 radicals selected from C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆-alkoxycarbonyl, —Si(R^{f})₂R^{g}, phenyl, benzyl, pyridyl and phenoxy, it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
or R^{c} and R^{d}, together with the nitrogen atom to which they are bound, form a 3-, 4-, 5-, 6- or 7-membered saturated, partly unsaturated or completely unsaturated N-heterocyclic ring which may contain 1 or 2 further heteroatoms selected from N, O and S as ring members, where the heterocyclic ring may carry 1, 2, 3 or 4 substituents selected from halogen, C₁-C₄-alkyl, C₁-C₄-haloalkyl, C₁-C₄-alkoxy and C₁-C₄-haloalkoxy,
R^{e} is selected from the group consisting of halogen, cyano, C₁-C₆-alkyl, C₂-C₆-alkenyl, C₂-C₆-alkynyl and C₃-C₈-cycloalkyl, where the four last-mentioned radicals may be unsubstituted, partially or fully halogenated and/or wherein one or two CH₂ groups may be replaced by a CO group, and/or may carry 1-2 radicals selected from C₁-C₄-alkoxy, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkoxy, C₁-C₆-alkylsulfonyl, C₁-C₆-haloalkylsulfonyl, C₁-C₆-alkoxycarbonyl, —Si(R^{f})₂R^{g}, phenyl, benzyl, pyridyl and phenoxy, it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
wherein, in the case of more than one R^{e}, R^{e} can be identical or different,
R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, C₃-C₈-cycloalkyl-C₁-C₄-alkyl, phenyl and benzyl,
R^{h}, R^{k} are, independently from one another, selected from the group consisting of hydrogen, halogen, cyano, azido, nitro, —SCN, SF₅, C₁-C₆-alkyl, C₂-C₆-alkenyl, C₂-C₆-alkynyl and C₃-C₈-cycloalkyl, where the four last mentioned radicals may be unsubstituted, partially or fully halogenated and/or oxygenated, and/or may carry 1 or 2 radicals selected from C₁-C₄-alkyl; C₁-C₄-haloalkyl; C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —Si(R^{f})₂R^{g}, —OH, —SH, phenyl, benzyl, pyridyl and phenoxy,
   it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy; (C₁-C₆-alkoxy)carbonyl, (C₁-C₆-alkyl)amino, di(C₁-C₆-alkyl)amino,
or R^{h} and R^{k} together form a group ═C(C₁-C₄-alkyl)₂, ═N(C₁-C₆-alkyl), ═NO(C₁-C₆-alkyl), or ═O,
R^{x} is selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl, said substituents R^{x} being identical or different from one another if more than one substituent R^{x} is present,
R^{y} is selected from the group consisting of hydrogen, cyano, C₁-C₆-alkyl, C₁-C₆-haloalkyl, C₁-C₆-alkoxy, C₁-C₆-haloalkoxy, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl and C₃-C₈-cycloalkyl-C₁-C₄-alkyl;
m is 1 or 2, wherein, in the case of several occurrences, m may be identical or different,
n is 0, 1 or 2; wherein, in the case of several occurrences, n may be identical or different,
   the process comprising the reaction of a sulfide of formula (II) with hydroxylamine-O-sulfonic acid of the formula (III),

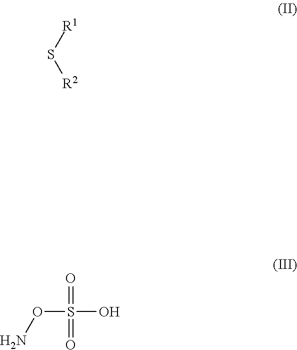

wherein R¹ and R² are as defined for formula (I),
   wherein the reaction is carried out in an aqueous medium in the presence of a base.

This process is hereinafter also referred to as “process A”.

The process A provides the sulfimine of the formula (Ia) or its salt of the formula (Ib) in high yields by reacting the sulfide of the formula (II) with the hydroxylamine-O-sulfonic acid of the formula (III) in an aqueous medium in the presence of a base. The use of the aqueous medium has the particular advantage that it substantially alleviates the hazard posed by hydroxylamine-O-sulfonic acid due to the high enthalpy of vaporization of water. In addition, it has been found that the aqueous reaction mixture obtained from the conversion of process A can directly be used in a follow-up reaction in which the sulfimine is acylated to a N-(2-amino-benzoyl)-sulfimine.

Accordingly, in a second aspect the present invention relates to a process for preparing a N-(2-amino-benzoyl)-sulfimine of the formula (IV),

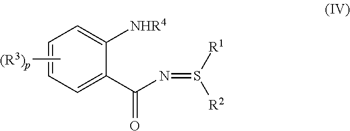

wherein
R³ if present, are independently selected from the group consisting of halogen, cyano, azido, nitro, —SCN, SF₅, C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₂-C₈-alkynyl, C₂-C₈-haloalkynyl, wherein the eight last radicals may optionally be substituted by one or more radicals R^{a}, —OR^{b}, SR^{b}, —S(O)ₘR^{b}, —S(O)ₙN(R^{c})R^{d}, —N(R^{c})R^{d}, —Si(R^{f})₂R^{g}, —N(R^{c})C(═O)R^{b}, —C(═NR^{c})R^{b}, —C(═O)N(R^{c})R^{d}, —C(═S)N(R^{c})R^{d}, phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e},
for p>1 it being possible that R³ are identical or different,
or two radicals R³ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—,—CH₂CH₂NR^{y}—, —CH₂CH═N—, —CH═CH—NR^{y}—, —CH═N—NR^{y}—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group,
R⁴ is selected from the group consisting of hydrogen, C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a}, phenyl which may be substituted by 1, 2, 3, 4 or 5 radicals R^{e}; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e},
p is 0, 1, 2, 3 or 4,
R¹, R², R^{a}, R^{b}, R^{c}, R^{d}, R^{e}, R^{f}, R^{g}, R^{h}, R^{k}, R^{y}, m and n are as defined herein above,
   the process comprising:
(i) providing a sulfimine of the formula (Ia), its salt of the formula (Ib) or a mixture thereof via process A,
(ii) reacting the sulfimine (Ia), its salt (Ib) or a mixture thereof obtained in step (i) with an isatoic acid anhydride of the formula (V) in the presence of a base,

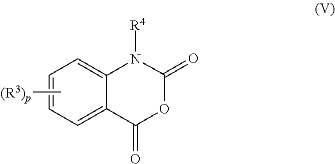

wherein R³, R⁴ and p are as defined above.

This process is hereinafter also referred to as “process B”.

The process B provides a N-(2-amino-benzoyl)-sulfimine of the formula (IV) in high yield by reacting the sulfimine of formula (Ia) and/or its salt of formula (Ib) obtained from process A with an isatoic anhydride of the formula (V) in the presence of a base. It has been found that the conversion of process B can be carried out in an aqueous medium. This is, on the one hand, surprising as hydroxide-initiated ring opening would have been expected to be the competing or even dominating reaction (see for example D. A. Clark et al, Bioorganic & Medicinal Chemistry 2008, 16, 3163). On the other hand, it allows the process B to be conducted as an one-pot process because an intermediate work-up procedure is not required and the reaction mixture obtained in step (i) can be directly introduced into step (ii). Hence, process B is highly economical and well suited for the production on an industrial scale.

In the context of the present invention, the terms used generically are defined as follows:

The prefix Cₙ-Cₘ indicates the number of possible carbon atoms in the particular case.

The term halogen denotes in each case fluorine, bromine, chlorine or iodine, in particular fluorine, chlorine or bromine.

The term “partially or fully halogenated” means that 1 or more, e.g. 1, 2, 3, 4 or 5 or all of the hydrogen atoms of a given radical have been replaced by a halogen atom, in particular by fluorine or chlorine.

The term “alkyl” as used herein (and in the alkyl moieties of other groups comprising an alkyl group, e.g. alkoxy, alkylcarbonyl, alkylthio, alkylsulfinyl, alkylsulfonyl and alkoxy-alkyl) denotes in each case a straight-chain or branched alkyl group having usually from 1 to 10 carbon atoms, frequently from 1 to 8 or from carbon atoms, preferably 1 to 4 carbon atoms and in particular from 1 to 3 carbon atoms. Examples of an alkyl group are methyl, ethyl, n-propyl, isopropyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl and 2-propylpentyl.

The term “haloalkyl” as used herein (and in the haloalkyl moieties of other groups comprising a haloalkyl group, e.g. haloalkoxy and haloalkylthio) denotes in each case a straight-chain or branched alkyl group having usually from 1 to 10 carbon atoms, frequently from 1 to 8 or from 1 to 6 carbon atoms, wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms, 3 to 8 carbon atoms or 3 to 6 carbon atoms, such as cyclopropyl, cyclobutyl, cyclopentyl, cyclohexyl, cycloheptyl, cyclooctyl, bicyclo[2.1.1]hexyl, bicyclo[3.1.1]heptyl, bicyclo[2.2.1]heptyl, and bicyclo[2.2.2]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, 3 to 8 carbon atoms or 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “alkenyl” as used herein denotes in each case a singly unsaturated hydrocarbon radical having usually 2 to 10, frequently from 2 to 8 or from 2 to 6 carbon atoms, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 10, frequently 2 to 8 or 2 to 6 carbon atoms, preferably 2 to 4 carbon atoms and one or two triple bonds in any position, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term haloalkynyl as used herein, which is also expressed as “alkynyl which may be substituted by halogen”, refers to unsaturated straight-chain or branched hydrocarbon radicals having usually 3 to 10 carbon atoms, frequently 2 to 6, preferably 2 to 4 carbon atoms, and one or two triple bonds in any position (as mentioned above), where some or all of the hydrogen atoms in these groups are replaced by halogen atoms as mentioned above, in particular fluorine, chlorine and bromine.

The term “cycloalkyl-alkyl” used herein denotes a cycloalkyl group, as defined above, which is bound to the remainder of the molecule via an alkylene group having preferably from 1 to 4 carbon atoms. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkylene” (or alkanediyl) as used herein in each case denotes an alkyl radical as defined above having usually from 2 to 9 or from 3 to 7 or from 3 to 5 carbon atoms, wherein one hydrogen atom at any position of the alkyl group is replaced by one further binding site, thus forming a bivalent moiety.

The term “alkenylene” (or alkenediyl) as used herein in each case denotes an alkenyl radical as defined above having usually from 2 to 9 or from 3 to 7 or from 3 to 5 carbon atoms, wherein one hydrogen atom at any position of the carbon backbone is replaced by one further binding site, thus forming a bivalent moiety.

The term “alkynylene” (or alkynediyl) as used herein in each case denotes an alkynyl radical as defined above having usually from 3 to 9 or from 3 to 7 or from 3 to 5 carbon atoms, wherein one hydrogen atom at any position of the carbon backbone is replaced by one further binding site, thus forming a bivalent moiety.

The term “alkoxy” as used herein denotes in each case a straight-chain or branched alkyl group usually having from 1 to 10 carbon atoms, frequently from 1 to 6 carbon atoms, preferably 1 to 4 carbon atoms, which is bound to the remainder of the molecule via an oxygen atom. Examples of an alkoxy group are methoxy, ethoxy, n-propoxy, isopropoxy, n-butyloxy, 2-butyloxy, iso-butyloxy, tert-butyloxy, and the like.

The term “haloalkoxy” as used herein denotes in each case a straight-chain or branched alkoxy group, as defined above, having from 1 to 10 carbon atoms, frequently from 1 to 6 carbon atoms, preferably 1 to 4 carbon atoms, preferably 1 to 3 carbon atoms, wherein the hydrogen atoms of this group are partially or totally replaced with halogen atoms, in particular fluorine atoms. Preferred haloalkoxy moieties include C₁-C₄-haloalkoxy, in particular halomethoxy, and also in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2-dichloro-2-fluorethoxy, 2,2,2-trichloroethoxy, pentafluoroethoxy and the like.

The term “alkoxy-alkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)butyl, 4-(2-methylpropoxy)-butyl, 4-(1,1-dimethylethoxy)-butyl and the like.

The term “alkylthio” (also alkylsulfanyl or alkyl-S—)” as used herein denotes in each case a straight-chain or branched saturated alkyl group as defined above, usually comprising 1 to 10 carbon atoms, frequently comprising 1 to 6 carbon atoms, preferably 1 to 4 carbon atoms, which is attached via a sulfur atom at any position in the alkyl group. Examples are methylthio, ethylthio, n-propylthio, isopropylthio, n-butylthio, 2-butylthio, iso-butylthio, tert-butylthio, and the like.

The term “haloalkylthio” as used herein refers to an alkylthio group as defined above wherein the hydrogen atoms are partially or fully substituted by fluorine, chlorine, bromine and/or iodine. Examples are fluoromethylthio, difluoromethylthio, trifluoromethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoro-ethylthio, 2,2-dichloro-2-fluorethylthio, 2,2,2-trichloroethylthio, pentafluoroethylthio and the like

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₆-alkylsulfinyl” refers to a C₁-C₆-alkyl group, as defined above, attached via a sulfinyl [S(O)] group. Examples are methylsulfinyl, ethylsulfinyl, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl), 1,1-dimethylethylsulfinyl (tert-butylsulfinyl), pentylsulfinyl, 1-methylbutylsulfinyl, 1,2-dimethylpropylsulfinyl, hexylsulfinyl, 1-methylpentylsulfinyl, 1,1-dimethylbutylsulfinyl, 1-ethylbutylsulfinyl, 1,1,2-trimethylpropylsulfinyl and 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. For example, the term “C₁-C₆-alkylsulfonyl” refers to a C₁-C₆-alkyl group, as defined above, attached via a sulfonyl [S(O)₂] group. Examples are methylsulfonyl, ethylsulfonyl, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl), 1,1-dimethylethylsulfonyl (tert-butylsulfonyl), pentylsulfonyl, 1-methylbutylsulfonyl, 1,1-dimethylpropylsulfonyl, 1-ethylpropylsulfonyl, hexylsulfonyl, 2-methylpentylsulfonyl, 1,1-dimethylbutylsulfonyl, 1-ethylbutylsulfonyl, 1,1,2-trimethylpropylsulfonyl and 1-ethyl-2-methylpropylsulfonyl.

The term “alkylamino” as used herein denotes in each case a group —NHR, wherein R is a straight-chain or branched alkyl group usually having from 1 to 6 carbon atoms, preferably 1 to 4 carbon atoms. Examples of an alkylamino group are methylamino, ethylamino, n-propylamino, isopropylamino, n-butylamino, 2-butylamino, isobutylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms, preferably 1 to 4 carbon atoms. Examples of a dialkylamino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, alkoxycarbonyl and haloalkoxycarbonyl.

The term “3-, 4-, 5-, 6-, 7- or 8-membered saturated, partially unsaturated or completely unsaturated heterocyclic ring containing 1, 2 or 3 heteroatoms or heteroatom groups selected from N, O, S, NO, SO and SO₂, as ring members” [wherein “completely/fully unsaturated” includes also “aromatic”] as used herein denotes monocyclic radicals, the monocyclic radicals being saturated, partially unsaturated or fully unsaturated (including aromatic) and which in addition to carbon atoms carry at least one, namely 1, 2 or 3 heteroatoms or heteroatom groups as ring members. The heterocyclic ring may be attached to the remainder of the molecule via a carbon ring member or via a nitrogen ring member. In the latter case, the heterocyclic ring is also termed as an N-heterocyclic ring.

Examples of a 3-, 4-, 5-, 6-, 7- or 8-membered saturated heterocyclic ring include: oxiranyl, aziridinyl, azetidinyl, tetrahydrofuran-2-yl, tetrahydrofuran-3-yl, tetrahydrothien-2-yl, tetrahydrothien-3-yl, pyrrolidin-2-yl, pyrrolidin-3-yl, pyrazolidin-3-yl, pyrazolidin-4-yl, pyrazolidin-5-yl, imidazolidin-2-yl, imidazolidin-4-yl, oxazolidin-2-yl, oxazolidin-4-yl, oxazolidin-5-yl, isoxazolidin-3-yl, isoxazolidin-4-yl, isoxazolidin-5-yl, thiazolidin-2-yl, thiazolidin-4-yl, thiazolidin-5-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl, morpholin-2-yl, morpholin-3-yl, thiomorpholin-2-yl, thiomorpholin-3-yl, 1-oxothiomorpholin-2-yl, 1-oxothiomorpholin-3-yl, 1,1-dioxothiomorpholin-2-yl, 1,1-dioxothiomorpholin-3-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

A 3-, 4-, 5-, 6-, 7- or 8-membered completely unsaturated (including aromatic) heterocyclic ring is e.g. a 5- or 6-membered fully unsaturated (including aromatic) heterocyclic ring. Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 4-isoxazolyl, 2-thiazolyl, 4-thiazolyl, 5-thiazolyl, 4-isothiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “3-, 4-, 5-, 6-, 7- or 8-membered saturated carbocyclic ring” as used herein refers to carbocyclic rings, which are monocyclic and fully saturated. Examples of such rings include cyclopropane, cyclobutane, cyclopentane, cyclohexane, cycloheptane, cyclooctane and the like.

The terms “3-, 4-, 5-, 6-, 7- or 8-membered partially unsaturated carbocyclic ring” and “5- or 6-membered partially unsaturated carbocyclic ring” refer to carbocyclic rings, which are monocyclic and have one or more degrees of unsaturation. Examples of such rings include cyclopropene, cyclobutene, cyclopentene, cyclohexene, cycloheptene, cyclooctene and the like.

The term “a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members” as used herein denotes a saturated or unsaturated 3- to 8-membered ring system which optionally contains 1 to 3 heteroatoms selected from N, O, S, NO, SO and SO₂, as defined above, with the exception of the completely unsaturated ring systems.

According to one embodiment of the invention in the processes A and B preference is given to the compounds of formulae (Ia), (Ib), (II) and (IV), where the variables R¹ and R², independently of each other, are selected from the group consisting of C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, wherein alkyl, alkenyl and cycloalkyl may optionally be substituted by one or more, e.g. 1 or 2 radicals R^{a}.

In this context R^{a} is preferably selected from the group consisting of cyano, SF₅, C₃-C₈-cycloalkyl, C₃-C₈-halocycloalkyl, —Si(R^{f})₂R^{g}, —OR^{b}, —SR^{b}, —S(O)ₘR^{b}, —S(O)ₙN(R^{c})R^{d}, —N(R^{c})R^{d}, —C(═O)N(R^{c})R^{d}, and phenyl which is unsubstituted or may be substituted by 1, 2, 3, 4 or 5 radicals R^{e}.

According to a particular embodiment of the invention in the processes A and B preference is given to the compounds of formulae (Ia), (Ib), (II) and (IV), where the variables R¹ and R², independently of each other, are selected from the group consisting of C₁-C₆-alkyl, C₃-C₇-cycloalkyl and C₃-C₈-cycloalkyl-C₁-C₄-alkyl.

According to another particular embodiment of the invention in the processes A and B preference is given to the compounds of formulae (Ia), (Ib), (II) and (IV), where the variables R¹ and R² together represent a C₃-C₇-alkylene or C₃-C₇-alkenylene group forming together with the sulfur atom to which they are attached a 4-, 5-, 6-, 7- or 8-membered, in particular a 5-, 6 or 7-membered, saturated or partially unsaturated ring, wherein 1 or 2 of the CH₂ groups in the C₃-C₇-alkylene chain or 1 or 2 of any of the CH₂ or CH groups in the C₃-C₇-alkenylene chain may be replaced by 1 or 2 groups independently selected from the group consisting of O, S and NR^{y}, and wherein the carbon atoms in the C₃-C₇-alkylene or C₃-C₇-alkenylene chain may be substituted with 1 to 5 identical or different substituents R^{x}, i.e. each of the carbon atoms may be unsubstituted or may carry 1 or 2 substituents R^{x} with a maximum of 5 substituents R^{x}, in particular with a maximum of 2 substituents R^{x} per alkylene or alkenylene chain. According to this particular embodiment of the invention R¹ and R² together preferably represent a C₄-C₇-alkylene group forming together with the sulfur atom to which they are attached a 5-, 6-, 7- or 8-membered, in particular a 5-, 6 or 7-membered, saturated ring.

In this context, R^{x} is preferably selected from the group consisting of halogen and C₁-C₄-alkyl, in particular from the group consisting of fluorine, chlorine and methyl, and R^{y} is preferably C₁-C₄-alkyl, in particular methyl.

In the processes A and B of the invention preference is further given to the compounds of formulae (IV) and (V), where one or more variables R³, if present, are independently selected from the group consisting of halogen, cyano, azido, nitro, —SCN, SF₅, C₁-C₈-alkyl, C₁-C₈-haloalkyl, C₃-C₈-cycloalkyl, C₃-C₈-halocycloalkyl, C₂-C₈-alkenyl and C₂-C₈-haloalkenyl, preferably from the group consisting of halogen, cyano, C₁-C₄-alkyl and C₁-C₄-haloalkyl, and in particular from the group consisting of halogen, in particular chlorine or bromine, methyl, cyano and halomethyl, e.g. trifluoromethyl, difluoromethyl or bromodifluoromethyl.

In this context, the variable p formulae (IV) and (V) is preferably 0, 1 or 2, in particular 1 or 2.

If present, i.e. if in formulae (IV) and (V) the variable p is ≠0, at least one radical R³ is preferably located in meta position with regard to the C(O) group.

In the processes A and B of the invention preference is further given to the compounds of formulae (IV) and (V), where the variable R⁴ is selected from the group consisting of hydrogen, C₁-C₆-alkyl, C₁-C₆-haloalkyl, C₃-C₇-cycloalkyl, C₃-C₇-halocycloalkyl, C₂-C₆-alkenyl and C₂-C₆-haloalkenyl, and in particular is hydrogen.

In the process B of the invention particular preference is given to compounds of the formulae (IV) and (V), where p is selected from 0, 1 or 2, and where for p=1 the one radical R³ is located in meta position with regard to the C(O) group, while for p=2, both radicals R³ are located in meta position with regard to the C(O) group. Particular preferred compounds of the formulae (IV) and (V) are represented by the following formulae (IVa) and (Va), respectively,

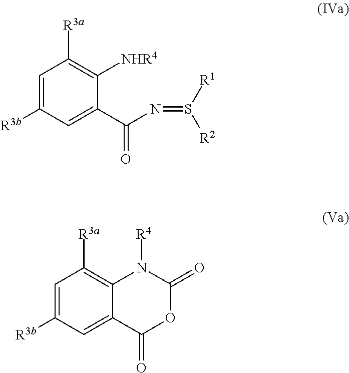

where R¹ and R² and R⁴ are as defined herein and where R^{3a} is hydrogen or has one of the meanings given herein for R³ and R^{3b} is hydrogen or has one of the meanings given herein for R³.

In formulae (IVa) and (Va) the radicals R^{3a} and R^{3b} are, independently of each other, preferably selected from the group consisting of hydrogen, halogen, C₁-C₄-alkyl, C₁-C₄-haloalkyl and cyano, it being possible that R^{3a} and R^{3b} are identical or different. In formulae (IVa) and (Va) the radical R^{3a} is in particular selected from the group consisting of hydrogen, halogen, in particular chlorine or bromine, methyl, and halomethyl, e.g. trifluoromethyl, difluoromethyl or bromodifluoromethyl, and the radical R^{3b} is in particular selected from the group consisting of hydrogen, halogen, in particular chlorine or bromine, cyano, methyl, and halomethyl, e.g. trifluoromethyl, difluoromethyl or bromodifluoromethyl. The radical R⁴ in formulae (IVa) and (Va) is in particular hydrogen.

Apart from that, the variables R^{a}, R^{b}, R^{c}, R^{d}, R^{e}, R^{f}, R^{g}, R^{h}, R^{k}, R^{x} and R^{y}, irrespectively of their occurrence, preferably have the following meanings, individually or in combination:
R^{a} selected from the group consisting of cyano, SF₅, C₃-C₈-cycloalkyl, C₃-C₈-halocycloalkyl, —Si(R^{f})₂R^{g}, —OR^{b}, —SR^{b}, —S(O)ₘR^{b}, —S(O)ₙN(R^{c})R^{d}, —N(R^{c})R^{d}, —C(═O)N(R^{c})R^{d}, and phenyl which is unsubstituted or may be substituted by 1, 2, 3, 4 or 5 radicals R^{e}, where R^{b}, R^{c}, R^{d}, R^{e}, R^{f} and R^{g} are as defined herein. R^{a} is preferably selected from the group consisting of cyano, C₃-C₈-cycloalkyl, C₁-C₄-alkoxy, C₁-C₄-alkylcarbonyl, C₁-C₄-alkoxycarbonyl, C₁-C₄-alkylthio, C₁-C₄-alkylsulfonyl, —S(O)ₙN(R^{c})R^{d}, —N(R^{c})R^{d} and —C(═O)N(R^{c})R^{d};
R^{b} selected from the group consisting of C₁-C₄-alkyl, C₁-C₄-haloalkyl, C₃-C₈-cycloalkyl, phenyl and benzyl;
R^{c} selected from the group consisting of hydrogen, C₁-C₄-alkyl, C₁-C₄-haloalkyl, C₁-C₄-alkoxy, C₃-C₈-cycloalkyl, C₃-C₈-cycloalkyl-CH₂, phenyl and benzyl;
R^{d} selected from the group consisting of hydrogen, cyano, C₁-C₄-alkyl, C₁-C₄-haloalkyl, C₁-C₄-alkoxy, C₃-C₈-cycloalkyl, C₃-C₈-cycloalkyl-CH₂, phenyl and benzyl;
R^{c}, R^{d} together with the nitrogen atom, to which they are bound may form a saturated 5-, 6- or 7-membered N-heterocycle, which may contain 1 or 2 further heteroatoms selected from N, O and S as ring members, where the heterocyclic ring may carry 1, 2, 3 or 4 substituents selected from C₁-C₄-alkyl;
R^{e} selected from the group consisting of halogen, in particular fluorine, chlorine or bromine, cyano, C₁-C₄-alkyl, C₁-C₄-haloalkyl, C₁-C₄-alkoxy, C₁-C₄-haloalkoxy and C₁-C₄-alkoxycarbonyl, especially from the group consisting of halogen, in particular fluorine, chlorine or bromine, cyano, methyl, methoxy, halomethyl, e.g. trifluoromethyl, difluoromethyl or bromodifluoromethyl, and halomethoxy, e.g. trifluoromethoxy, difluoromethoxy or fluoromethoxy;
R^{f} C₁-C₄-alkyl, in particular methyl;
R^{g} C₁-C₄-alkyl, in particular methyl, C₅-C₆-cycloalkyl, C₁-C₄-alkoxy-C₁-C₄-alkyl, C₅-C₆-cycloalkyl-CH₂ and phenyl;
R^{h} selected from the group consisting of hydrogen, halogen, cyano and C₁-C₆-alkyl;
R^{k} selected from the group consisting of hydrogen, halogen, cyano and C₁-C₆-alkyl;
R^{x} selected from the group consisting of halogen and C₁-C₄-alkyl, in particular from the group consisting of fluorine chlorine and methyl.
R^{y} C₁-C₄-alkyl, in particular methyl.

In the processes A and B of the invention specific preference is given to compounds of the formulae (Ia), (Ib) and (II), where R¹ and R², independently of each other, are selected from the group consisting of C₁-C₄-alkyl, and in particular form the group consisting of methyl, ethyl and isopropyl.

In the process B of the invention specific preference is given to compounds of the formulae (IVa) and (Va), where:
R¹ and R², if present, are independently of each other selected from the group consisting of C₁-C₄-alkyl, and in particular form the group consisting of methyl, ethyl and isopropyl;
R^{3a} is selected from the group consisting of methyl and halogen, and in particular form the group consisting of methyl, chlorine and bromine;
R^{3b} is selected from the group consisting of cyano, methyl and halogen, and in particular form the group consisting of cyano, chlorine and bromine; and
R⁴ is hydrogen.

According to a specific embodiment of the invention in the process B preference is given to the compounds of formula (IVa) in which R⁴ is hydrogen and the combination of R¹, R², R^{3a} and R^{3b} for a compound corresponds in each case to the meanings given for these variables in one of the rows A-1 to A-45 of Table A (compounds IVa-1 to IVa-45).

According to another specific embodiment of the invention in the process B preference is given to the compounds of formula (Va) in which R⁴ is hydrogen and the combination of R^{3a} and R^{3b} for a compound corresponds in each case to the meanings given for these variables in one of the rows A-1 to A-45 of Table A (compounds Va-1 to Va-45).

TABLE A

| | R¹ | R² | R^{3a} | R^{3b} |
|---|---|---|---|---|
| A-1 | methyl | methyl | methyl | chlorine |
| A-2 | ethyl | methyl | methyl | chlorine |
| A-3 | isopropryl | methyl | methyl | chlorine |
| A-4 | methyl | ethyl | methyl | chlorine |
| A-5 | ethyl | ethyl | methyl | chlorine |
| A-6 | isopropryl | ethyl | methyl | chlorine |
| A-7 | methyl | isopropryl | methyl | chlorine |
| A-8 | ethyl | isopropryl | methyl | chlorine |
| A-9 | isopropryl | isopropryl | methyl | chlorine |
| A-10 | methyl | methyl | chlorine | chlorine |
| A-11 | ethyl | methyl | chlorine | chlorine |
| A-12 | isopropryl | methyl | chlorine | chlorine |
| A-13 | methyl | ethyl | chlorine | chlorine |
| A-14 | ethyl | ethyl | chlorine | chlorine |
| A-15 | isopropryl | ethyl | chlorine | chlorine |
| A-16 | methyl | isopropryl | chlorine | chlorine |
| A-17 | ethyl | isopropryl | chlorine | chlorine |
| A-18 | isopropryl | isopropryl | chlorine | chlorine |
| A-19 | methyl | methyl | methyl | cyano |
| A-20 | ethyl | methyl | methyl | cyano |
| A-21 | isopropryl | methyl | methyl | cyano |
| A-22 | methyl | ethyl | methyl | cyano |
| A-23 | ethyl | ethyl | methyl | cyano |
| A-24 | isopropryl | ethyl | methyl | cyano |
| A-25 | methyl | isopropryl | methyl | cyano |
| A-26 | ethyl | isopropryl | methyl | cyano |
| A-27 | isopropryl | isopropryl | methyl | cyano |
| A-28 | methyl | methyl | chlorine | bromine |
| A-29 | ethyl | methyl | chlorine | bromine |
| A-30 | isopropryl | methyl | chlorine | bromine |
| A-31 | methyl | ethyl | chlorine | bromine |
| A-32 | ethyl | ethyl | chlorine | bromine |
| A-33 | isopropryl | ethyl | chlorine | bromine |
| A-34 | methyl | isopropryl | chlorine | bromine |
| A-35 | ethyl | isopropryl | chlorine | bromine |
| A-36 | isopropryl | isopropryl | chlorine | bromine |
| A-37 | methyl | methyl | bromine | bromine |
| A-38 | ethyl | methyl | bromine | bromine |
| A-39 | isopropryl | methyl | bromine | bromine |
| A-40 | methyl | ethyl | bromine | bromine |
| A-41 | ethyl | ethyl | bromine | bromine |
| A-42 | isopropryl | ethyl | bromine | bromine |
| A-43 | methyl | isopropryl | bromine | bromine |
| A-44 | ethyl | isopropryl | bromine | bromine |
| A-45 | isopropryl | isopropryl | bromine | bromine |

N-(2-Amino-benzoyl)-sulfimines of the formula (IV) are particularly suitable as precursors for the preparation of N-(het)arylpyrazole carboxanilides carrying a sulfiminocarbonyl group in the ortho position on the anilide moiety. These compounds are known for example from WO 2007/006670 and WO 2013/024008. They can advantageously be prepared in accordance to the process disclosed in WO 2013/024008 using the compounds of formula (IV) obtainable via the process B of the present invention.

A further aspect of the present invention relates to a process for preparing a compound of formula (VI),

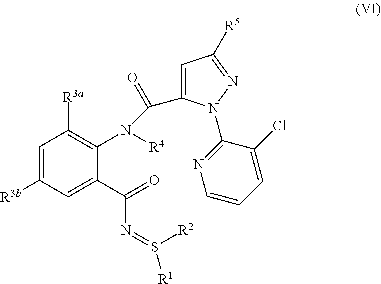

wherein R¹, R², R^{3a}, R^{3b} and R⁴ are as defined herein, and R⁵ is selected from halogen, C₁-C₄-haloalkyl and C₁-C₄-alkoxy, in particular from CF₃, CHF₂ and CCl₃, which process comprises reacting a compound of the formula (IVa) that is prepared by process B of the invention, with a pyrazole compound of the formula (VII),

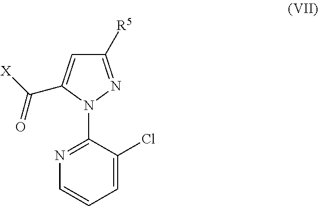

wherein X is a suitable leaving group, such as in particular hydroxyl or halogen, and R⁵ is as defined above. The reaction can be carried out by analogy to conventional amidation reactions of carboxylic acids, activated carboxylic acids or carboxylic acid chlorides with aromatic amines as described e.g. in WO 2003/015519, WO 2006/062978, WO 2008/07158, WO 2009/111553 or WO 2013/076092. The reaction is preferably carried out according to the procedure described in WO 2013/024008, and may for example be conducted in the following manner: a solution or a suspension of a base, such an alkalimetal carbonate or a tertiary amine, and the compound of formula (IV) in a suitable aprotic organic solvent is charged to a reaction vessel. To this mixture, an equimolar or almost equimolar amount of the compound of formula (VII), where X is halogen, in particular chlorine, is added, preferably as a solution or suspension in an organic solvent. To the resulting mixture, an amidation catalyst, such as 4-(N,N-dimethylamino)pyridine, may be added, if desired. The catalyst may be added in an amount of 0.005 to 0.2 mol, preferably 0.01 to 0.1 mol per mol of compound of formula (VII), either neat, in solution or as a suspension in a suitable organic solvent. The reaction is generally performed at a temperature from 0 to 110° C. and preferably at a temperature from 30 to 80° C.

The compounds of formula (VII) are known from the prior art, in particular from WO 2003/015519, WO 2013/024008 and WO 2013/076092, and can be prepared by analogy to methods described therein.

The reactions of the invention as described hereinafter are performed in reaction vessels customary for such reactions, the reaction being carried out in a continuous, semi-continuous or batchwise manner. In general, the particular reactions will be carried out under atmospheric pressure. The reactions may, however, also be carried out under reduced or elevated pressure.

The reaction of process A according to the invention for preparing a sulfimine of the formula (Ia) or its salt of the formula (Ib), or a mixture thereof may be regarded as a S-amination. The conversion is effected by reacting a sulfide of the formula (II), such as in particular a thioether, with hydroxylamine-O-sulfonic acid of the formula (III) in an aqueous medium in the presence of a base.

In the reaction of process A the hydroxylamine-O-sulfonic acid (III) is preferably used in an amount of 0.6 to 2.5 mol, more preferably of 0.8 to 2.0 mol, even more preferably of 1.0 to 1.5 mol, especially of 1.0 to 1.2 mol and in particular of 1.0 to 1.1 mol, based in each case on 1 mol of the sulfide of formula (II).

In the reaction of process A the base may be used in catalytic or stoichiometric amounts. Preferably, the base is used in an amount of 0.1 to 2.5 mol, more preferably of 0.8 to 2.0 mol, even more preferably of 1.0 to 1.5 mol, especially of 1.0 to 1.2 mol and in particular of 1.0 to 1.1 mol, based in each case on 1 mol of the sulfide of formula (II).

Suitable bases for the reaction of process A include oxo bases and organic bases.

Suitable oxo bases are, for example, alkali metal and alkaline earth metal hydroxides, such as lithium hydroxide (LiOH), sodium hydroxide (NaOH), potassium hydroxide (KaOH) and calcium hydroxide (Ca(OH)₂), alkali metal and alkaline earth metal alkoxides, especially sodium and potassium alkanolates, such as sodium methoxide, potassium methoxide, sodium ethoxide, potassium ethoxide, sodium tert-butanolate, potassium tert-butanolate, sodium 2-methylbutan-2-olate and potassium 2-methylbutan-2-olate, alkali metal phosphates, such as trisodium phosphate and tripotassium phosphate, alkali metal hydrogenphosphates, such as disodium hydrogenphosphate and dipotassium hydrogenphosphate, alkali metal and alkaline earth metal carbonates, such as lithium carbonate, potassium carbonate and calcium carbonate, and also alkali metal bicarbonates, such as sodium bicarbonate. Preference is given to an aqueous NaOH solution or an aqueous KOH solution.

Suitable organic bases are advantageously selected from organic amine bases, i.e. bases wherein the site of basicity is a nitrogen atom. Preferably, the amine base is a tertiary alkyl-, alkenyl-, or alkinylamine or an arylamine or a heterocyclic aromatic amine. Preference is given to trimethylamine, triethylamine, dimethylcyclohexylamine, diisopropylethylamine and tri-n-butylamine, N-methyl pyrrolidine, N-methyl piperidine, N-methyl morpholine, N,N′-dimethyl piperazine, DABCO (1,4-diazabicyclo[2.2.2]-octane), DBU (1,8-diazabicyclo[5.4.0]undec-7-ene), DBN (1,5-diazabicyclo[4.3.0]non-5-ene), pyridine, 2-picoline, 3-picoline, 2-ethylpyridine, 2,3-lutidine, 2,4-lutidine, 2,5-lutidine, 2,6-lutidine, 3,4-lutidine and 3,5-lutidine.

According to one embodiment of the invention the base used in the reaction of process A is selected from alkali metal hydroxides and organic amine bases, and preferably from alkali metal hydroxides. According to a preferred embodiment of the invention NaOH and in particular an aqueous solution of NaOH is used as the base.

The aqueous medium used in process A is selected from water and mixtures of water with an organic solvent as co-solvent that is preferably fully miscible with water. The amount of organic solvent usually is less than 50% by volume, preferably is less than 20% by volume and in particular is less than 10% by volume based on the total amount of the aqueous medium.

Suitable organic solvents in this respect should be sufficiently inert under the reaction conditions. Suitable water-miscible organic solvents may be selected from tetrahydrofurane (THF), acetonitrile, dioxane, acetone, C₁-C₃-alkanoles; such as methanol, ethanol, n-propanol, isopropanol, tert-butanol or 2-methylbutan-2-ol, butanone, dimethylformamide (DMF), dimethylacetamide (DMAc), N-methyl-2-pyrrolidone (NMP), dimethyl sulfoxide (DMSO) and mixtures thereof. Preference is given here to THF, acetone, tert-butanol, butanone, acetonitrile, dioxane or a mixture thereof.

According to a particular embodiment of the invention the reaction of process A is carried out in an aqueous medium that consists or at least predominately consists of water, i.e. does not include organic solvent or at least does not include a substantial amount of organic solvent.

The total amount of the aqueous medium used in the reaction of process A according to the invention is typically in the range from 200 to 3000 g, preferably in the range from 250 to 1500 g and in particular in the range of 400 to 1200 g, based in each case on 1 mol of the sulfide of the formula (II).

The reactants can in principle be contacted with one another in any desired sequence. For example, the hydroxylamine-O-sulfonic acid of the formula (III) and the sulfide of the formula (II), if appropriate in dissolved or dispersed form, can be initially charged and mixed with each other. The obtained mixture is then admixed with the base. Conversely, the base, if appropriate in dissolved or dispersed form, can be initially charged and admixed with a mixture of the hydroxylamine-O-sulfonic acid (III) and the sulfide (II). Alternatively, all reactants can also be added simultaneously to the reaction vessel. As an alternative to the joint addition of the hydroxylamine-O-sulfonic acid (III) and the sulfide (II) they can also be added separately to the reaction vessel. Both of them can independently of one another be added, either in a solvent or in bulk, before or after the addition of the base. However, in general it is preferable to avoid contacting the hydroxylamine-O-sulfonic acid (III) with the base in the absence of the sulfide (II).

It has been found to be beneficial to initially charge the reaction vessel with the hydroxylamine-O-sulfonic acid (III), preferably in dispersed or dissolved form, more preferably in dissolved form and in particular as dissolved in water or in the aqueous medium, and admix the sulfide (II) in bulk or in a solvent which is selected from the aqueous medium, water and the aforementioned organic solvent. The sulfide (II) is preferably admixed by gradually adding it in bulk or in a solvent, particularly in bulk, to the reaction vessel. To the thus obtained mixture comprising the hydroxylamine-O-sulfonic acid (III) and the sulfide (II) in the aqueous medium is added the base which is employed as such or in dissolved or dispersed form. The base may be added in one portion or gradually, with the gradual addition being preferred.

In general, the reaction of process A is performed under temperature control. The reaction is typically effected in a closed or preferably in an open reaction vessel with stirring apparatus. In general, during the conversion of process A the temperature of the reaction mixture is kept at values not higher than 80° C., preferably not higher than 60° C., more preferably not higher than 50° C. and especially not higher than 40° C., e.g. the temperature is kept in the range from 0 to 80° C., preferably in the range from 10 to 60° C., more preferably in the range from 15 to 50° C. and specifically in the range from 20 to 40° C.

According to a preferred embodiment of the invention the reaction of process A is initiated by starting to gradually add the base to a mixture of the hydroxylamine-O-sulfonic acid (III) and the sulfide (II) at a lower temperature of typically below 50° C., preferably below 40° C., more preferably below 30° C. and especially below 25° C. The addition of the base is continued in such a way that the temperature does not exceed 80° C., preferably not exceed 60° C. and in particular not exceed 40° C. After completion of the base addition the temperature is usually maintained in the range of 10 to 55° C., preferably in the range from 15 to 40° C. and specifically in the range from 18 to 30° C.

Depending on the solvent used, on the reaction temperature and on whether the reaction vessel possesses a vent, a pressure of generally 1 to 5 bar and preferably of 1 to 3 bar is established during the reaction.

After the conversion of process A is completed or at least has sufficiently proceeded the obtained reaction mixture may be used as is in a following reaction step or may be subjected to a work-up procedure.

If desired, the work-up of the reaction mixtures obtained in the reaction of process A can be effected in a customary manner, e.g. by removing the solvent, for example under reduced pressure. Depending in particular on the organic co-solvent potentially included in the aqueous medium, it may also be possible to isolate the sulfimine of the formula (Ia) or its salt of the formula (Ib), or a mixture thereof, from the reaction medium via crystallization or precipitation, preferably after having removed insoluble byproducts. Precipitation or crystallization may be achieved by concentrating and/or cooling the reaction mixture. The isolated product can be further purified, e.g. by crystallization or tituration with a solvent, e.g. with acetonitrile. Frequently, however, the product obtained at this stage is already of sufficient purity and further purification steps are not required.

According to a preferred embodiment of the invention the reaction mixture obtained from the conversion of process A, or from the conversion in step (i) of process B, respectively, is directly introduced in a subsequent reaction step, such as in particular step (ii) of process B, without any prior work-up step.

The process B according to the invention for preparing a N-(2-amino-benzoyl)sulfimine of the formula (IV) comprises the steps (i) and (ii). In step (i) a sulfimine of the formula (Ia), its salt of the formula (Ib) or a mixture thereof is provided by the process A of the present invention, as described herein above. In step (ii) the sulfimine (Ia) and/or its salt (Ib) obtained in step (i) is converted into the N-(2-amino-benzoyl)-sulfimine (IV) by reaction with an isatoic anhydride of the formula (V) in the presence of a base. This reaction may be regarded a N-acylation.

The conversion in step (ii) of process B can be carried out in an organic solvent in accordance to procedures disclosed in WO 2013/024008, or, alternatively, in an aqueous medium.

According to preferred embodiment of the invention the conversion in step (ii) of process B is carried out in an aqueous medium which is selected from water and mixtures of water with an organic solvent as co-solvent. Suitable organic solvents in this respect should be sufficiently inert under the reaction conditions and may be miscible with water, i.e. form homogeneous mixtures with water in all proportions, or may be immiscible with water, i.e. do not form homogeneous mixtures with water in all proportions. Thus, depending on whether an organic co-solvent is used and on whether the optional organic co-solvent is water-miscible or water-immiscible, the conversion in step (ii) is carried out in a homogeneous or in a biphasic solvent system.

Water-miscible organic solvents that are suitable as co-solvent in step (ii) of process B may be selected from THF, acetonitrile, dioxane, acetone, C₁-C₃-alkanoles, such as methanol, ethanol, n-propanol or isopropanol, butanone, DMF, DMAc, NMP, DMSO and mixtures thereof, and are preferably selected from THF, acetone, butanone, acetonitrile, dioxane and mixtures thereof. In case such a water-miscible organic solvent is used as co-solvent it is usually present in the aqueous medium in an amount of less than 60% by volume, preferably less than 40% by volume and in particular less than 20% by volume, based on the total amount of the aqueous medium.

Water-immiscible organic solvents that are suitable as co-solvent in step (ii) of process B are preferably selected from those having a high polarity, such as for example dichloromethane, chloroform, 1,2-dichloroethane, toluene, benzene, ortho-xylene, para-xylene, meta-xylene, chlorobenzene, methyl isobutyl ketone, 2-methyltetrahydrofuran (2-Me-THF), ethyl acetate, n-propyl acetate, n-butyl acetate, ethyl propionate, diethyl ether, diisopropyl ether and methyl tert-butyl ether (MTBE). Preference is given here to 2-Me-THF, MTBE, ethyl acetate, n-propyl acetate, n-butyl acetate, toluene, benzene, chlorobenzene. 1,2-dichloroethane, dichloromethane, chloroform and mixtures thereof. In case such a water-immiscible organic solvent is used as co-solvent it is usually present in the aqueous medium in an amount of from 30 to 90% by volume, preferably from 40 to 85% by volume, more preferably from 45 to 80% by volume, and in particular from 50 to 75% by volume, based on the total amount of the aqueous medium.

Particularly preferred co-solvents for use in the conversion in step (ii) of process B are selected from THF, acetone, butanone, acetonitrile, dioxane, 2-Me-THF, MTBE, ethyl acetate, n-propyl acetate, n-butyl acetate, dichloromethane, 1,2-dichloroethane, chloroform, benzene, chlorobenzene, toluene and mixtures thereof, and especially selected from 2-Me-THF, ethyl acetate, n-butyl acetate, 1,2-dichloroethane and mixtures thereof.

According to a preferred embodiment of the invention the conversion in step (ii) of process B is carried out in an aqueous medium that includes as co-solvent an organic solvent, which is preferably selected from the organic solvents mentioned herein as preferred.

The total amount of the aqueous medium used in step (ii) of process B according to the invention is typically in the range from 500 to 8000 g, preferably in the range from 800 to 4000 g and in particular in the range of 1000 to 3000 g, based in each case on 1 mol of the sulfimine of the formula (Ia), its salt of the formula (Ib), or a mixture thereof.

In step (ii) of process B the isatoic anhydride (V) is preferably used in an amount of 0.6 to 1.3 mol, more preferably of 0.8 to 1.15 mol, even more preferably of 0.85 to 1.1 mol and especially of 0.9 to 1.05 mol, based in each case on 1 mol of the sulfimine of the formula (Ia), its salt of the formula (Ib), or a mixture thereof.

In step (ii) of process B the base is preferably used in an amount of 0.5 to 1.5 mol, more preferably of 0.7 to 1.25 mol, even more preferably of 0.8 to 1.15 mol and especially of 0.85 to 1.1 mol, based in each case on 1 mol of the sulfimine of the formula (Ia), its salt of the formula (Ib), or a mixture thereof.

Suitable bases for the reaction in step (ii) of process B are typically selected from the oxo bases and organic bases mentioned in the context of process A, preferably from the aforementioned alkali metal hydroxides and organic amine bases and more preferably from alkali metal hydroxides. In this respect particular preference is given to NaOH and specifically to an aqueous solution of NaOH.

According to a particular embodiment of the invention the base used in step (ii) of the process B is the same as the one used in step (i), i.e. in the process A.

In step (ii) of process B the reactants can in principle be contacted with one another in any desired sequence. For example, the sulfimine (Ia) and/or its salt (Ib) and the isatoic anhydride (V), if appropriate in dissolved or dispersed form, can be initially charged and mixed with each other. The obtained mixture is then admixed with the base. Conversely, the base, if appropriate in dissolved or dispersed form, can be initially charged and admixed with a mixture of the sulfimine (Ia) and/or its salt (Ib) and the isatoic anhydride (V). Alternatively, all reactants can also be added simultaneously to the reaction vessel. As an alternative to the joint addition of the sulfimine (Ia) and/or its salt (Ib) and the isatoic anhydride (V) they can also be added separately to the reaction vessel. Both of them can independently of one another be added, either in a solvent or in bulk, before or after the addition of the base. However, in case the base is an oxo-base isatoic anhydride (V) should not be contacted with the base in the absence of the sulfimine (Ia) and/or its salt (Ib).

According to a particular embodiment of the invention in step (ii) of process B at first the isatoic anhydride (V), either in bulk or in dispersed or dissolved form, is contacted and admixed directly with the reaction mixture of the conversion in step (i) of process B, i.e. the reaction mixture of process A. Here, the reaction mixture of step (i) is employed as is without any prior work-up procedure.

According to a particular preferred embodiment of the invention the conversion in step (ii) comprises the following substeps:
(a) adding the isatoic anhydride (V) to the reaction mixture of the conversion in step (i) in accordance with the aforementioned particular embodiment, and then
(b) gradually adding the base to the reaction mixture obtained in substep (a) so as to maintain the pH of the reaction mixture at a value not higher than 13, preferably not higher than 12, more preferably not higher than 11 and in particular not higher than 10.

In substep (a) the isatoic anhydride (V) is added in bulk or in dispersed or dissolved form, preferably in bulk or as dispersed or dissolved in an organic solvent. In case the isatoic anhydride (V) is added in dispersed or dissolved form the respective dispersion or solution is preferably prepared by using the complete or the partial volume of the organic co-solvent to be used in step (ii) according to the aforementioned preferred embodiment of the invention. It is particularly preferred that in substep (a) the isatoic anhydride (V) is added in bulk and the organic co-solvent is added before or after the addition of the isatoic anhydride (V).

In substep (b) the base is preferably employed as such or preferably in dissolved or dispersed form, e.g. in the form of an aqueous solution in case NaOH is used as base. The gradual addition of the base can be effected e.g. by a constant rate of addition which allows to keep the pH of the reaction mixture at a value not exceeding 13, preferably 12, more preferably 11 and in particular 10, as described herein before.

In the event the process B of the invention is conducted, in accordance to the aforementioned particular embodiment, as an one-pot process by adding the isatoic anhydride (V) directly to the reaction mixture obtained in step (i), the amounts of isatoic anhydride (V) and base to be used in step (ii) may be calculated on the basis of the amount of the sulfide of formula (II) employed in step (i) as follows:

Isatoic anhydride (V) is preferably used in an amount of 0.7 to 1.2 mol, more preferably of 0.8 to 1.1 mol and especially of 0.9 to 1.0 mol, while the base is preferably used in an amount of 0.7 to 1.3 mol, more preferably of 0.8 to 1.2 mol and especially of 0.9 to 1.1 mol, based in each case on 1 mol of sulfide (II).

Typically, after completion of the base addition the conversion in step (ii) of process B is continued until the sulfimine (Ia) and/or its salt (Ib) is entirely or almost entirely consumed.

In general, the conversion in step (ii) of process B is performed under temperature control. The reaction is typically effected in a closed or preferably in an open reaction vessel with stirring apparatus. In general, during the conversion of process B the temperature of the reaction mixture is kept at values not higher than 80° C., preferably not higher than 70° C., more preferably not higher than 50° C. and especially not higher than 45° C., e.g. the temperature is kept in the range from 0 to 80° C., preferably in the range from 5 to 70° C., more preferably in the range from 10 to 50° C. and specifically in the range from 15 to 45° C.

Depending on the solvent used, on the reaction temperature and on whether the reaction vessel possesses a vent, a pressure of generally 1 to 5 bar and preferably of 1 to 3 bar is established during the reaction.

The work-up of the reaction mixture obtained from the conversion in step (ii) of process B and the isolation of the N-(2-amino-benzoyl)-sulfimine of the formula (IV) are effected in a customary manner, for example by extraction with a suitable solvent. Suitable solvents for this purpose are the aforementioned water-immiscible, polar organic solvents capable of dissolving the N-(2-amino-benzoyl)-sulfimines (IV). Thus, in case such a solvent was used as co-solvent in step (ii) and the reaction is conducted in a biphasic solvent system, the organic phase can simply be separated from the aqueous phase which may optionally be extracted again with said water-immiscible organic solvent. In case a water-miscible organic solvent was used as co-solvent, it may be necessary to concentrate the reaction mixture, at least to some extent, in order to completely or partially remove the water-miscible organic solvent, and then resuspend the obtained residue in a mixture of water and said water-immiscible, polar organic solvent. In both cases the combined organic phases obtained may optionally be washed one or more times with a suitable aqueous medium, e.g. water or an aqueous acidic solution, such as aqueous sodium hydrogencarbonate, dried and then concentrated to dryness, e.g. under reduced pressure, to yield the crude product. Alternatively, in particular in cases where no co-solvent or a water-miscible organic solvent was used, the work-up of the reaction mixtures can be effected by concentrating the reaction mixture to dryness and isolating the crude product via crystallization or precipitation from a suitable solvent, or, alternatively, via trituration with a suitable solvent.

The thus obtained crude product can be further purified, e.g. by crystallization or by chromatography or combined measures. However, frequently, the crude product is already obtained in a purity which does not require further purification steps.

### EXAMPLES

The compounds were characterized by quantitative High Performance Liquid Chromatography (HPLC) employing the following procedure:

Analytical HPLC column: RP-18 column Chromolith Speed ROD from Merck KgaA (Germany). Elution: acetonitrile+0.1% trifluoroacetic acid (TFA)/water+0.1% trifluoroacetic acid (TFA) in a ratio of from 5:95 to 95:5 in 5 minutes at 40° C.

Detection: ESI-MS, positive ion mode.

### Example 1

### 2-Amino-5-chloro-N-(diethyl-λ⁴-sulfanylidene)-3-methyl-benzamide (use of 1,2 dichloroethane as organic co-solvent)

2.30 g (20.32 mmol, 1.27 eq.) Hydroxylamine-O-sulfonic acid were dissolved in 14 ml demineralized water (pH of the solution: 0.4). 1.84 g Diethyl sulfide (20.35 mmol, 1.27 eq.) were added within 1 min at 20° C. Then 3.25 g of aqueous NaOH (25% by weight, 20.29 mmol, 1.27 eq.) were added within 22 min at 24-30° C. (pH of the reaction mixture: 3.4). After 2.5 hours of stirring at 23° C., 3.60 g (16.00 mmol, 1.00 eq., purity: 94%) 6-chloro-8-methyl-1H-3,1-benzoxazine-2,4-dione (prepared by analogy to the method of example P.2 of WO2013/024008) and 32 ml 1,2 dichloroethane were added. After 10 min further 3.25 g of aqueous NaOH (25% by weight, 20.31 mmol, 1.27 eq.) were added within 35 minutes at 23° C. (pH of the reaction mixture: 9.5 to max. 10.1). Stirring was continued for 18 h at 23° C. and afterwards for 4 h at 40° C. (pH of the reaction mixture: 7.5). The phases were separated at 23° C. and analyzed by quantitative HPLC. The organic phase (37.6 g) contained 9.8% by weight of the title compound (13.53 mmol, yield: 84.6%).

### Example 2

### 2-Amino-5-chloro-N-(diethyl-λ⁴-sulfanylidene)-3-methyl-benzamide (use of butyl acetate as organic co-solvent)

23.53 g (208.02 mmol, 1.30 eq.) Hydroxylamine-O-sulfonic acid were dissolved in 140 ml demineralized water (pH of the solution: 0.2). 18.76 g Diethyl sulfide (208.02 mmol, 1.30 eq.) were added within 1 min at 20° C. Then 33.25 g of aqueous NaOH (25% by weight, 207.83 mmol, 1.30 eq.) were added within 30 min at 23-37° C. (pH of the reaction mixture: 3.4). After 2.5 hours stirring at 23° C., 36.02 g (160.01 mmol, 1.00 eq., purity: 94%) 6-chloro-8-methyl-1H-3,1-benzoxazine-2,4-dione and 320 ml butyl acetate were added. After 20 min at 23-33° C. further 33.30 g of aqueous NaOH (25% by weight, 208.14 mmol, 1.30 eq.) were added within 80 min at 33° C. (pH of the reaction mixture: 9.0 to max. 9.8). The reaction mixture was stirred for 10 h at 33° C. (pH of the reaction mixture: 7.3). Then another 1.50 g of aqueous NaOH (25% by weight, 9.38 mmol, ca. 0.06 eq.) were added. The reaction mixture was stirred for 3 h at 33° C. (pH of the reaction mixture: 7.8). The duff was filtered and the phases were separated at 23° C. The organic layer was washed four times with 220 ml of aqueous sodium bicarbonate (5% by weight). Quantitative HPLC revealed the organic layer (294.4 g) to contain 13.28% by weight of the title compound (143.33 mmol, yield: 89.6%).

### Example 3

### 2-Amino-5-chloro-N-(diethyl-λ⁴-sulfanylidene)-3-methyl-benzamide (use of ethyl acetate as organic co-solvent)

2.53 g (22.40 mmol, 1.40 eq.) Hydroxylamine-O-sulfonic acid were dissolved in 14 ml demineralized water (pH of the solution: 0.8). 2.01 g Diethyl sulfide (22.24 mmol, 1.39 eq.) were added within 1 min at 20° C. Then 3.62 g of aqueous NaOH (25% by weight, 22.63 mmol, 1.41 eq.) were added within 23 min at 22-30° C. (pH of the reaction mixture: 6.1). After stirring for 2 h at 23° C. 3.39 g (16.00 mmol, 1.00 eq., purity: 100%) 6-chloro-8-methyl-1H-3,1-benzoxazine-2,4-dione and 30 ml ethyl acetate were added. After 10 min at 23° C. further 3.25 g of aqueous NaOH (25% by weight, 20.31 mmol, 1.27 eq.) were added within 60 min at 23° C. (pH of the reaction mixture: 9 to max. 9.5). The reaction mixture was stirred for 16 h at 23° C. (pH of the reaction mixture: 7.6) and then heated to 40° C. for 1 h (pH of the reaction mixture: 7.7). The phases were separated at 23° C. The organic layer was washed with 30 ml of aqueous sodium bicarbonate (5% by weight) three times. The organic layer was dried over sodium sulfate, filtered and concentrated in vacuum (140 mbar/35° C.) to yield 15.21 g. Quantitative HPLC revealed the organic phase to contain 22.68% by weight of the title compound (12.63 mmol, yield: 78.9%).

### Example 4

### 2-Amino-5-chloro-N-(diethyl-λ⁴-sulfanylidene)-3-methyl-benzamide (use of ethyl acetate as organic co-solvent)

1.81 g (16.00 mmol, 1.00 eq.) Hydroxylamine-O-sulfonic acid were dissolved in 14 ml demineralized water (pH of the solution: 0.8). 1.44 g Diethyl sulfide (16.00 mmol, 1.00 eq.) were added within 1 min at 20° C. Then 2.57 g of aqueous NaOH (25% by weight, 16.04 mmol, 1.00 eq.) were added within 10 min at 18-26° C. (pH 3.6). After 2 h stirring at 23° C., 3.39 g (16.00 mmol, 1.00 eq., purity: 100%) 6-chloro-8-methyl-1H-3,1-benzoxazine-2,4-dione and 30 ml ethyl acetate were added. After 10 min at 23° C. further 2.56 g of aqueous NaOH (25% by weight, 16.00 mmol, 1.00 eq.) were added within 80 min at 23° C. (pH of the reaction mixture: 9 to max. 10.4). The reaction mixture was stirred for 64 h at 23° C. (pH of the reaction mixture: 7.4) and then heated to 40° C. (pH 7.1) for 7.5 h. The phases were separated at 23° C. and washed with 30 ml of aqueous sodium bicarbonate (5% by weight) three times. The organic phase was dried over sodium sulfate, filtered and the filter cake was washed with ethyl acetate. Quantitative HPLC revealed the combined organic phases (28.6 g) to contain 11.64% by weight of the title compound (12.18 mmol, yield: 76.2%).

### Example 5

### 2-Amino-5-chloro-N-(diethyl-λ⁴-sulfanylidene)-3-methyl-benzamide (use of 2-methyltetrahydrofuran as organic co-solvent)

2.53 g (22.40 mmol, 1.40 eq.) Hydroxylamine-O-sulfonic acid were dissolved in 14 ml demineralized water (pH of the solution: 0.8). 2.02 g Diethyl sulfide (22.40 mmol, 1.40 eq.) were added within 1 min at 20° C. Then 3.61 g of aqueous NaOH (25% by weight, 22.59 mmol, 1.41 eq.) were added within 25 min at 23-31° C. (pH of the reaction mixture: 4.8). After 2.5 h of stirring at 23° C. 3.39 g (16.00 mmol, 1.00 eq., purity: 100%) 6-chloro-8-methyl-1H-3,1-benzoxazine-2,4-dione and 33 ml 2-methyltetrahydrofuran were added. After 10 min at 23° C. further 3.25 g of aqueous NaOH (25% by weight, 20.31 mmol, 1.27 eq.) were added within 50 min at 23° C. (pH of the reaction mixture: 8.7 to max. 9.1). The reaction mixture was stirred for 16 h at 23° C. (pH of the reaction mixture: 7.5) and then heated to 40° C. for 0.75 h (pH of the reaction mixture: 7.6). The phases were separated at 23-40° C. and washed with 30 ml of aqueous sodium bicarbonate (5% by weight) three times. The organic phase was dried over sodium sulfate, filtered and the filter cake washed with 2-methyltetrahydrofuran. Quantitative HPLC revealed the combined organic phases (28.10 g) to contain 12.62% by weight of the title compound (12.98 mmol, yield: 81.1%).

### Example 6

### 2-Amino-5-chloro-N-(diethyl-λ⁴-sulfanylidene)-3-methyl-benzamide (use of toluene as organic co-solvent)

2.17 g (19.21 mmol, 1.20 eq.) Hydroxylamine-O-sulfonic acid were dissolved in 14 ml demineralized water (pH of the solution: 0.8). 1.73 g Diethyl sulfide (19.20 mmol, 1.20 eq.) were added within 1 min at 20° C. Then 3.07 g of aqueous NaOH (25% by weight, 19.20 mmol, 1.20 eq.) were added within 15 min at 24-30° C. (pH of the reaction mixture: 3.6). After 2.5 h of stirring at 23° C., 3.39 g (16.00 mmol, 1.00 eq., purity: 100%) 6-chloro-8-methyl-1H-3,1-benzoxazine-2,4-dione and 40 ml toluene were added. After 10 min at 23° C. further 3.08 g of aqueous NaOH (25% by weight, 19.25 mmol, 1.20 eq.) were added within 150 min at 23° C. (pH of the reaction mixture: 9.5 to max. 11). The reaction mixture was stirred for 16 h at 23° C. (pH of the reaction mixture: 8.7) and then heated to 40° C. for 13 h (pH of the reaction mixture: 7.6). The phases were separated at 23° C. and the organic phase was washed with 30 ml of aqueous sodium bicarbonate (5% by weight) three times. Quantitative HPLC revealed the organic phase (33.30 g) to contain 8.59% by weight of the title compound (10.48 mmol, yield: 65.5%).

### Example 7

### 2-Amino-5-chloro-N-(diethyl-λ⁴-sulfanylidene)-3-methyl-benzamide (use of xylene as organic co-solvent)

2.53 g (22.40 mmol, 1.40 eq.) Hydroxylamine-O-sulfonic acid were dissolved in 14 ml demineralized water (pH of the solution: 0.8). 2.03 g Diethyl sulfide (22.51 mmol, 1.41 eq.) were added within 1 min at 20° C. Then 3.61 g of aqueous NaOH (25% by weight, 22.58 mmol, 1.41 eq.) were added within 13 min at 23-29° C. (pH of reaction mixture: 5.5). After 3.5 h of stirring at 23° C. 3.39 g (16.00 mmol, 1.00 eq., purity: 100%) 6-chloro-8-methyl-1H-3,1-benzoxazine-2,4-dione and 30 ml xylene were added. After 10 min at 23° C. further 3.24 g of aqueous NaOH (25% by weight, 20.25 mmol, 1.27 eq.) were added within 80 min at 23° C. (pH of the reaction mixture: 9 to max. 10.7). The reaction mixture was stirred for 16 h at 23° C. (pH of the reaction mixture: 8.5) and then heated to 40° C. for 5 h (pH of the reaction mixture: 7.6). The phases were separated at 40° C. and washed with 30 ml of aqueous sodium bicarbonate (5% by weight) three times. The organic phase was filtered (duff), distilled four times with 10 ml xylene and concentrated in vacuum. Quantitative HPLC revealed the oily organic phase (3.02 g) to contain 87.87% by weight of the title product (9.72 mmol, yield: 60.7%).

By analogy to the methods of examples 1 to 7 the following compounds of formula IV, wherein R⁴ is H can be prepared:

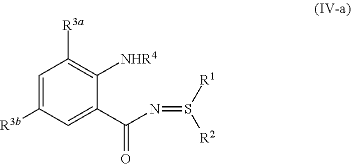

| R¹ | R² | R^{3a} | R^{3b} | R⁴ | HPLC/MS (Method) |
|---|---|---|---|---|---|
| CH(CH₃)₂ | CH(CH₃)₂ | Cl | Cl | H | 3.346 min, m/z = 321.05 (A) |
| C₂H₅ | C₂H₅ | Cl | Cl | H | 2.821 min, m/z = 292.9 (A) |
| CH₂-c-Pr | CH₂-c-Pr | CH₃ | Cl | H | 1.191 min, m/z = 325.5 (B) |
| CH₂-c-Pr | CH₂-c-Pr | Cl | Cl | H | 1.391 min, m/z = 320.8 (B) |
| CH₂-c-Pr | C₂H₅ | CH₃ | Cl | H | 1.197 min, m/z = 299.1 (B) |
| CH₂-c-Pr | CH(CH₃)₂ | Cl | Cl | H | 3.200 min, m/z = 333.0 (A) |
| CH₂-c-Pr | CH(CH₃)₂ | CH₃ | Cl | H | 2.433 min, m/z = 313.0 (A) |
| C₂H₅ | C₂H₅ | CF₃ | Cl | H | 3.218 min, m/z = 327.00 (A) |
| C₂H₅ | C₂H₅ | CF₃ | Br | H | 3.291 min, m/z = 372.90 (A) |
| C₂H₅ | C₂H₅ | Br | Cl | H | 2.980 min, m/z = 338.90 (A) |
| C₂H₅ | C₂H₅ | Cl | Br | H | 2.970 min, m/z = 338.90 (A) |
| CH(CH₃)₂ | CH(CH₃)₂ | CF₃ | Cl | H | 3.604 min, m/z = 355.05 (A) |
| CH(CH₃)₂ | CH(CH₃)₂ | CF₃ | Br | H | 3.677 min, m/z = 400.95 (A) |
| CH(CH₃)₂ | CH(CH₃)₂ | Br | Cl | H | 3.390 min, m/z = 366.95 (A) |
| CH(CH₃)₂ | CH(CH₃)₂ | Cl | Br | H | 3.381 min, m/z = 366.95 (A) |
| CH(CH₃)₂ | CH(CH₃)₂ | Br | Br | H | 3.409 min, m/z = 410.90 (A) |
| CH₂CH₂CH₃ | CH₂CH₂CH₃ | CH₃ | Cl | H | 1.046 min, m/z = 301.1 (B) |
| CH₂CH₂CH₃ | CH₂CH₂CH₃ | Cl | Cl | H | 3.441 min, m/z = 320.95 (A) |
| C₂H₅ | C₂H₅ | Br | Br | H | 1.102 min, m/z = 383.0 (B) |
| CH(CH₃)₂ | CH(CH₃)₂ | CH₃ | Cl | H | 2.510 min, m/z = 301.05 (A) |

CH₂-c-Pr = CH₂-cyclopropyl

### Preparation of the Compounds of Formula VI (Examples 8a and 8b)

### Example 8a

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide

To a suspension of potassium carbonate (0.71 g, 10 mmol, 1.3 equiv) and 2-amino-3-methyl-5-chloro-N-(diethyl-λ⁴-sulfanylidene)benzamide (1.42 g, 3.96 mmol, prepared as described above) in propylene carbonate (20 mL) was added a solution of 2-(3-chloro-2-pyridyl)-5-(trifluoromethyl)pyrazole-3-carbonyl chloride (1.35 g, 4.35 mmol, 1.10 equiv., prepared by the method described in WO2013/024008) in propylene carbonate (10 mL) at room temperature. After 24 h at this temperature, the mixture was poured onto water and spiked with ethanol under vigorous stirring. The resulting solids were collected by filtration and contained pure title compound (1.57 g, 73%).

LCMS (Method B): r.t. 1.19 min, m/z 546.1 (M+H)⁺; m.p. 189° C.;

¹H NMR (500 MHz, DMSO) [delta]: 10.87 (s, 1H), 8.53 (d, 1H), 8.22 (d, 1H), 7.75 (s, 1H), 7.65 (m, 2H), 7.40 (s, 1H), 3.09 (m, 2H), 2.92 (m, 2H) 1.15 (m, 6H).

### Example 8b

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide

To a solution of 2-(3-chloro-2-pyridyl)-5-(trifluoromethyl)pyrazole-3-carbonyl chloride (150 g, 435 mmol) in acetonitrile (900 mL) at room temperature potassium carbonate (59 g, 427 mmol) was added. A solution of 2-amino-5-chloro-N-(diethyl-sulfanylidene)-3-methyl-benzamide (117 g, 427 mmol, prepared as described in example 1) in acetonitrile (100 mL) was added dropwise within 1 hour while maintaining a reaction temperature of 25-28° C. with occasional cooling (slightly exothermic reaction). The mixture was stirred for 16 hours at room temperature. The reaction mixture was then poured on ice-water mixture (5 L) and the pH was adjusted to 7-8 with concentrated HCl. The mixture stirred for an additional 2 hours. The light brown solid was filtered, washed with water and dried under air to give the crude product (229 g).

3 combined batches of crude product (789 g) were suspended in acetonitrile (2.6 L) and dissolved upon heating at 60° C. After 1 hour of stirring at 60° C. the solution was cooled by means of an ice-bath and the thereby formed solid was filtered off. The mother-liquor was concentrated to 300 mL and cooled with ice-bath. Thereby additional solid formed was filtered. The combined solids were washed with cold acetonitrile and dried at 50° C. in a vacuum-oven over night to give the title product (703 g, 89%) as a crystalline white solid.

By analogy to the methods described for Examples 8a and 8b, the following compounds of formula VI can be prepared:

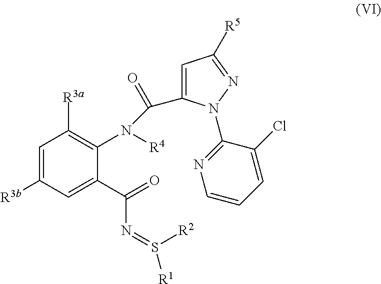

| R¹ | R² | R^{3a} | R^{3b} | R⁵ | HPLC/MS - Log P |
|---|---|---|---|---|---|
| CH(CH₃)₂ | CH(CH₃)₂ | Cl | Cl | CF₃ | 3.890 min*; m/z = 596.00 |
| CH₃ | CH₃ | Cl | Cl | CF₃ | 3.372 min*; m/z = 539.95 |
| CH₂CH₂CH₂CH₂ | | Cl | Cl | CF₃ | 3.543 min*; m/z = 564.00 |
| CH(CH₃)₂ | CH(CH₃)₂ | CH₃ | Cl | CF₃ | 3.704 min*; m/z = 574.00 |
| CH₂CH₂CH₂CH₂ | | CH₃ | Cl | CF₃ | 3.478 min*; m/z = 544.05 |
| C₂H₅ | C₂H₅ | Br | Cl | CF₃ | 3.633 min*; m/z = 611.85 |
| CH(CH₃)₂ | CH(CH₃)₂ | Br | Cl | CF₃ | 3.630 min*; m/z = 639.90 |
| C₂H₅ | C₂H₅ | Br | Br | CF₃ | 1.127 min**; m/z = 655.9 |
| CH(CH₃)₂ | CH(CH₃)₂ | Br | Br | CF₃ | 3.665 min*; m/z = 683.90 |
| C₂H₅ | C₂H₅ | CF₃ | Cl | CF₃ | 1.231 min**; m/z = 600.0 |
| CH(CH₃)₂ | CH(CH₃)₂ | CF₃ | Cl | CF₃ | 1.169 min**; m/z = 628.1 |
| C₂H₅ | C₂H₅ | CF₃ | Br | CF₃ | 1.248 min**; m/z = 645.9 |
| CH(CH₃)₂ | CH(CH₃)₂ | CF₃ | Br | CF₃ | 1.308 min**; m/z = 673.9 |
| C₂H₅ | C₂H₅ | Br | CF₃ | CF₃ | 1.301 min**; m/z = 646.1 |
| CH(CH₃)₂ | CH(CH₃)₂ | Br | CF₃ | CF₃ | 1.350 min**; m/z = 673.9 |
| C₂H₅ | C₂H₅ | Cl | CF₃ | CF₃ | 1.284 min**; m/z = 673.9 |
| CH(CH₃)₂ | CH(CH₃)₂ | Cl | CF₃ | CF₃ | 1.358 min**; m/z = 600.1 |
| C₂H₅ | C₂H₅ | Cl | CN | CF₃ | 1.171 min**; m/z = 557.3 |
| CH(CH₃)₂ | CH(CH₃)₂ | Cl | CN | CF₃ | 1.262 min**; m/z = 585.3 |
| C₂H₅ | C₂H₅ | CH₃ | CN | CF₃ | 1.179 min**; m/z = 537.3 |
| CH(CH₃)₂ | CH(CH₃)₂ | CH₃ | CN | CF₃ | 1.253 min**; m/z = 565.3 |
| CH₂CH₂CH₂CH₂ | | CH₃ | Cl | Br | 3.277 min*; m/z = 556.0 |
| CH₃ | CH₃ | CH₃ | Cl | Br | 3.067 min*; m/z = 529.9 |
| C₂H₅ | C₂H₅ | CH₃ | Cl | Br | 3.309 min*; m/z = 557.9 |
| CH(CH₃)₂ | CH(CH₃)₂ | CH₃ | Cl | Br | Log P: 2.9 [pH = 10.0] |
| CH₂CH₂CH₂CH₂ | | Cl | Cl | Br | 3.184 min*; m/z = 575.8 |
| CH₃ | CH₃ | Cl | Cl | Br | 3.015 min*; m/z = 549.8 |
| CH(CH₃)₂ | CH(CH₃)₂ | Cl | Cl | Br | 3.538 min*; m/z = 605.8 |
| CH₃ | CH₃ | CH₃ | Cl | CHF₂ | 1.060 min**; m/z = 500.2 |
| C₂H₅ | C₂H₅ | CH₃ | Cl | CHF₂ | 1.134 min**; m/z = 528.2 |
| CH(CH₃)₂ | CH(CH₃)₂ | CH₃ | Cl | CHF₂ | 1.225 min**; m/z = 556.3 |
| CH₃ | CH₃ | Cl | Cl | CHF₂ | 1.062 min**; m/z = 520.2 |
| C₂H₅ | C₂H₅ | Cl | Cl | CHF₂ | 1.144 min**; m/z = 549.9 |
| CH(CH₃)₂ | CH(CH₃)₂ | Cl | Cl | CHF₂ | 1.240 min**; m/z = 578.0 |
| C₂H₅ | C₂H₅ | Br | Cl | CHF₂ | 1.148 min**; m/z = 594.1 |
| CH(CH₃)₂ | CH(CH₃)₂ | Br | Cl | CHF₂ | 1.205 min**; m/z = 622.2 |
| C₂H₅ | C₂H₅ | Br | Br | CHF₂ | 1.171 min**; m/z = 638.1 |
| CH(CH₃)₂ | CH(CH₃)₂ | Br | Br | CHF₂ | 1.245 min**; m/z = 666.1 |
| C₂H₅ | C₂H₅ | CH₃ | CN | CHF₂ | 3.035 min*; m/z = 519.0 |
| CH(CH₃)₂ | CH(CH₃)₂ | CH₃ | CN | CHF₂ | 3.277 min*; m/z = 547.1 |

*Analytical HPLC column: RP-18 column Chromolith Speed ROD from Merck KgaA, Germany). Elution: acetonitrile + 0.1% trifluoroacetic acid (TFA)/water + 0.1% trifluoroacetic acid (TFA) in a ratio of from 5:95 to 95:5 in 5 minutes at 40° C.
**Analytical UPLC column: Phenomenex Kinetex 1.7 μm XB-C18 100A; 50 × 2.1 mm; mobile phase: A: water + 0.1% trifluoroacetic acid (TFA); B: acetonitrile + 0.1% TFA; gradient: 5-100% B in 1.50 minutes; 100% B 0.20 min; flow: 0.8-1.0 mL/min in 1.50 minutes at 60° C.
***logP determinations were performed via capillary electrophorese on a ce-Pro9600 ™ from CombiSep.

1-21. (canceled)

22. A process for preparing a compound of the formulae (Ia) or (Ib), or a mixture thereof, 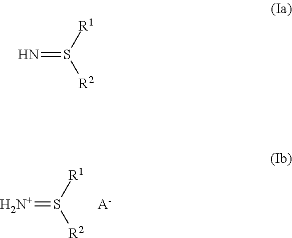
wherein
R¹ and R² are selected, independently of one another, from the group consisting of hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, and C₂-C₁₀-haloalkynyl, wherein the eight last radicals may optionally be substituted by one or more radicals R^{a},
or R¹ and R² together represent a C₂-C₉-alkylene, C₂-C₉-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₉-alkylene chain or 1 to 4 of any of the CH₂ or CH groups in the C₂-C₉-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NR^{y}, and wherein the carbon atoms in the C₂-C₉-alkylene, C₂-C₉-alkenylene or C₆-C₉-alkynylene chain may be substituted with 1 to 5 identical or different substituents R^{x}, and wherein the sulfur and nitrogen atoms in the C₂-C₉-alkylene, C₂-C₉-alkenylene or C₆-C₉-alkynylene chain, independently of one another, may be oxidized,
A⁻ is HSO₄⁻ or 1/2 SO₄²⁻,
R^{a} is selected from the group consisting of cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R^{f})₂R^{g}, —OR^{b}, —SR^{b}, —S(O)ₘR^{b}, —S(O)ₙN(R^{c})R^{d}, —N(R^{c})R^{d}, C(═O)R^{b}, C(═O)OR^{b}, C(═O)N(R^{c})R^{d}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from the group consisting of N, O, S, NO, SO and SO₂, as ring members, where the heterocyclic ring may be substituted by one or more radicals R^{e},
or two geminally bound radicals R^{a} together form a group selected from the group consisting of ═cR^{h}R^{k}, ═NR^{c}, ═NOR^{b} and ═NNR^{c},
or two radicals R^{a}, together with the carbon atoms to which they are bound, form a 3-, 4-, 5-, 6-, 7- or 8-membered saturated or partially unsaturated carbocyclic ring or a 3-, 4-, 5-, 6-, 7- or 8-membered saturated or partially unsaturated heterocyclic ring containing 1, 2 or 3 heteroatoms or heteroatom groups selected from the group consisting of N, O, S, NO, SO and SO₂, as ring members,
wherein, in the case of more than one R^{a}, R^{a} can be identical or different,
R^{b} is selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, where the five last mentioned radicals may be unsubstituted, partially or fully halogenated and/or wherein one or two CH₂ groups may be replaced by a CO group; and/or may carry 1-2 radicals selected from C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxycarbonyl, —Si(R^{f})₂R^{g}, phenyl, benzyl, pyridyl and phenoxy,
it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
wherein, in the case of more than one R^{b}, R^{b} can be identical or different,
R^{c}, R^{d} are, independently from one another, selected from the group consisting of hydrogen, cyano, C₁-C₆-alkyl, C₂-C₆-alkenyl, C₂-C₆-alkynyl, C₃-C₈-cycloalkyl, and C₃-C₈-cycloalkyl-C₁-C₄-alkyl, where the five last mentioned radicals may be unsubstituted, partially or fully halogenated and/or wherein one or two CH₂ groups may be replaced by a CO group; and/or may carry 1 or 2 radicals selected from the group consisting of C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆-alkoxycarbonyl, —Si(R^{f})₂R^{g}, phenyl, benzyl, pyridyl and phenoxy, it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
or R^{c} and R^{d}, together with the nitrogen atom to which they are bound, form a 3-, 4-, 5-, 6- or 7-membered saturated, partly unsaturated or completely unsaturated N-heterocyclic ring which may contain 1 or 2 further heteroatoms selected from the group consisting of N, O and S as ring members, where the heterocyclic ring may carry 1, 2, 3 or 4 substituents selected from the group consisting of halogen, C₁-C₄-alkyl, C₁-C₄-haloalkyl, C₁-C₄-alkoxy and C₁-C₄-haloalkoxy,
R^{e} is selected from the group consisting of halogen, cyano, C₁-C₆-alkyl, C₂-C₆-alkenyl, C₂-C₆-alkynyl and C₃-C₈-cycloalkyl, where the four last-mentioned radicals may be unsubstituted, partially or fully halogenated and/or wherein one or two CH₂ groups may be replaced by a CO group, and/or may carry 1-2 radicals selected from the group consisting of C₁-C₄-alkoxy, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxycarbonyl, —Si(R^{f})₂R^{g}, phenyl, benzyl, pyridyl and phenoxy,
it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy and C₁-C₆-alkoxycarbonyl,
wherein, in the case of more than one R^{e}, R^{e} can be identical or different,
R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, C₃-C₈-cycloalkyl-C₁-C₄-alkyl, phenyl and benzyl,
R^{h}, R^{k} are, independently from one another, selected from the group consisting of hydrogen, halogen, cyano, azido, nitro, —SCN, SF₅, C₁-C₆-alkyl, C₂-C₆-alkenyl, C₂-C₆-alkynyl and C₃-C₈-cycloalkyl, where the four last mentioned radicals may be unsubstituted, partially or fully halogenated and/or oxygenated, and/or may carry 1 or 2 radicals selected from the group consisting of C₁-C₄-alkyl; C₁-C₄-haloalkyl; C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —Si(R^{f})₂R^{g}, —OH, —SH, phenyl, benzyl, pyridyl and phenoxy,
it being possible for phenyl, benzyl, pyridyl and phenoxy to be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy; (C₁-C₆-alkoxy)carbonyl, (C₁-C₆-alkyl)amino, and di-(C₁-C₆-alkyl)amino,
or R^{h} and R^{k} together form a group ═C(C₁-C₄-alkyl)₂, ═N(C₁-C₆-alkyl), ═NO(C₁-C₆-alkyl), or ═O,
R^{x} is selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl, said substituents R^{x} being identical or different from one another if more than one substituent R^{x} is present,
R^{y} is selected from the group consisting of hydrogen, cyano, C₁-C₆-alkyl, C₁-C₆-haloalkyl, C₁-C₆-alkoxy, C₁-C₆-haloalkoxy, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl and C₃-C₈-cycloalkyl-C₁-C₄-alkyl;
m is 1 or 2, wherein, in the case of several occurrences, m may be identical or different,
n is 0, 1 or 2; wherein, in the case of several occurrences, n may be identical or different,
the process comprising the reaction of a sulfide of formula (II) with hydroxylamine-O-sulfonic acid of formula (III), 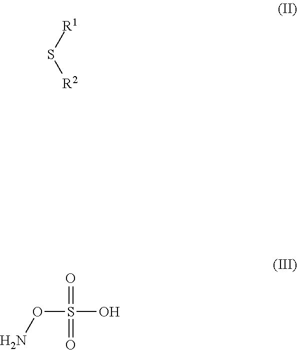
wherein
R¹ and R² are as defined for formula (I),
wherein the reaction is carried out in an aqueous medium in the presence of a base.

23. The process of claim 22, wherein R¹ and R², independently of one another, are selected from the group consisting of C₁-C₆-alkyl, C₁-C₆-haloalkyl, C₂-C₆-alkenyl, C₃-C₇-cycloalkyl and C₃-C₈-cycloalkyl-C₁-C₄-alkyl or
R¹ and R² together represent a C₃-C₇-alkylene or C₃-C₇-alkenylene forming together with the sulfur atom to which they are attached a 4-, 5-, 6-, 7- or 8-membered saturated or partially unsaturated ring, wherein 1 or 2 of the CH₂ groups in the C₃-C₇-alkylene chain or 1 or 2 of any of the CH₂ or CH groups in the C₃-C₇-alkenylene chain may be replaced by 1 or 2 groups independently selected from the group consisting of O, S and NR^{y}, and wherein the carbon atoms in the C₃-C₇-alkylene or C₃-C₇-alkenylene chain may be substituted with 1 to 5 identical or different substituents R^{x}.

24. The process of claim 22, wherein the base is added to a mixture comprising the sulfide of formula (II), the hydroxylamine-O-sulfonic acid of formula (III) and the aqueous medium.

25. The process of claim 22, wherein the base is selected from alkaline hydroxides and organic amines.

26. The process of claim 25, wherein the base is selected from alkaline hydroxides.

27. The process of claim 22, wherein the aqueous medium is water containing less than 10% by volume of organic solvent, based on the total amount of the aqueous medium.

28. The process of claim 22, wherein the hydroxylamine-O-sulfonic acid of formula (III) is used in an amount of 0.8 to 2 mol, based on 1 mol of the sulfide of formula (II).

29. The process claim 22, wherein the base is used in an amount of 0.8 to 2 mol, based in each case on 1 mol of the sulfide of formula (II).

30. The process of claim 22, wherein the temperature of the reaction mixture is maintained at a value not higher than 60° C.

31. A process for preparing a compound of the formula (IV), 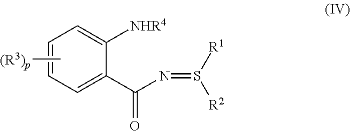
wherein
R³ if present, are independently selected from the group consisting of halogen, cyano, azido, nitro, —SCN, SF₅, C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₂-C₈-alkynyl, and C₂-C₈-haloalkynyl, wherein the eight last radicals may optionally be substituted by one or more radicals R^{a}, —OR^{b}, SR^{b}, —S(O)ₘR^{b}, —S(O)ₙN(R^{c})R^{d}, —N(R^{c})R^{d}, —Si(R^{f})₂R^{g}, —N(R^{c})C(═O)R^{b}, —C(═NR^{c})R^{b}, —C(═O)N(R^{c})R^{d}, —C(═S)N(R^{c})R^{d},
phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R^{e},
for p>1 it being possible that R³ are identical or different,
or two radicals R³ bound on adjacent carbon atoms may be together a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR^{y}—, —CH₂CH═N—, —CH═CH—NR^{y}—, —CH═N—NR^{y}—, —OCH═N— and —SCH═N—, thus forming, together with the carbon atoms to which they are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group,
R⁴ is selected from the group consisting of hydrogen, C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, C₂-C₁₀-alkynyl, and C₂-C₁₀-haloalkynyl, wherein the eight last radicals may optionally be substituted by one or more radicals R^{a},
phenyl which may be substituted by 1, 2, 3, 4 or 5 radicals R^{e}; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R^{e},
p is 0, 1, 2, 3 or 4,
R¹, R², R^{a}, R^{b}, R^{c}, R^{d}, R^{e}, R^{f}, R^{g}, R^{h}, R^{k}, R^{y}, m and n are as defined in claim 22,
the process comprising:
preparing a compound of the formulae (Ia) or (Ib), or a mixture thereof, by the process of claim 22,
(ii) reacting the compound of the formulae (Ia) or (Ib), or a mixture thereof, obtained in step (i) with a compound of the formula (V) in the presence of a base, 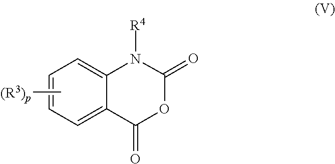
wherein R³, R⁴ and p are as defined above.

32. The process of claim 31, wherein R³, if present, are independently selected from the group consisting of halogen, cyano, azido, nitro, —SCN, SF₅, C₁-C₈-alkyl, C₁-C₈-haloalkyl, C₃-C₈-cycloalkyl, C₃-C₈-halocycloalkyl, C₂-C₈-alkenyl and C₂-C₈-haloalkenyl, and R⁴ is selected from the group consisting of hydrogen, C₁-C₆-alkyl, C₁-C₆-haloalkyl, C₃-C₇-cycloalkyl, C₃-C₇-halocycloalkyl, C₂-C₆-alkenyl and C₂-C₆-haloalkenyl.

33. The process of claim 32, wherein R⁴ in formulae (IV) and (V) is hydrogen.

34. The process of claim 31, wherein the base used in step (ii) is the same as the one used in step (i).

35. The process of claim 31, wherein the reaction mixture of the conversion in step (i) is introduced into step (ii) without prior work-up.

36. The process of claim 35, wherein step (ii) comprises
(a) adding to the reaction mixture of the conversion in step (i) the compound of the formula (V), and
(b) gradually adding the base so as to maintain the pH of the reaction mixture at a value not higher than 13.

37. The process of claim 36, wherein in step (a) the compound of the formula (V) is added either in solid form or as dissolved or dispersed in an organic solvent.

38. The process of claim 37, wherein the organic solvent is selected from the group consisting of THF, 2-Me-THF, MTBE, ethyl acetate, n-propyl acetate, n-butyl acetate, acetonitrile, dioxane, acetone, butanone, dichloromethane, 1,2-dichloroethane, chloroform, benzene, chlorobenzene and toluene.

39. The process of claim 31, wherein 0.8 to 1.1 mol of the compound of the formula (V) and 0.8 to 1.2 mol of the base are used, in each case based on 1 mol of the sulfide of formula (II).

40. The process of claim 31, wherein in step (ii) the temperature of the reaction mixture is maintained at a value not higher than 70° C.

41. A process for preparing a compound of the formula (VI), 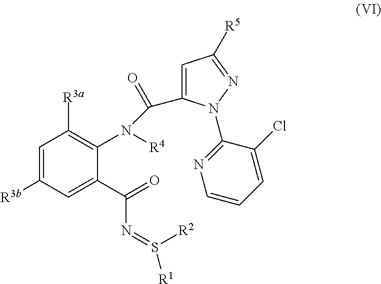
wherein
R⁵ is selected from the group consisting of halogen, C₁-C₄-haloalkyl and C₁-C₄-alkoxy, and
R¹, R², R^{3a}, R^{3b} and R⁴ are as defined in claim 31,
the process comprising:
(a) preparing a compound of the formula (IV) by the process of claim 31,
(b) reacting the compound of the formula (IV) obtained in step (a) with a compound of the formula (VII), 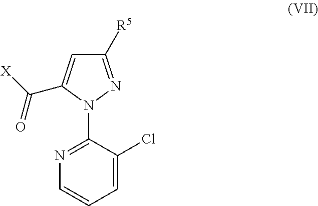
wherein X is selected from hydroxyl and halogen, and R⁵ is as defined above.

42. The process of claim 41, wherein R⁵ is selected from the group consisting of CF₃, CHF₂ and CCl₃.
